(19) United States
(12) Patent Application Publication
(10) Pub. No.: US 2022/0192585 A1
(43) Pub. Date: Jun. 23, 2022

(54) SYSTEM AND METHOD FOR PRECISE DETERMINATION OF A DATE OF CHILDBIRTH WITH A WEARABLE DEVICE

(71) Applicant: AVA AG, Zürich (CH)

(72) Inventors: Aljosa BILIC, Zürich (CH); Mohaned SHILAIH, Zürich (CH); Lisa FALCO, Zürich (CH)

(21) Appl. No.: 17/595,380

(22) PCT Filed: May 15, 2020
(86) PCT No.: PCT/EP2020/063598

§ 371 Date: Nov. 15, 2021

(30) Foreign Application Priority Data
May 16, 2019 (EP) 19020334.9

(51) Int. Cl. A61B 5/00; A61B 5/024

(52) U.S. Cl. CPC A61B 5/4343; A61B 5/681; A61B 5/02405; A61B 5/4812; A61B 5/02444; A61B 5/02438

(57) ABSTRACT

The invention relates to an electronic system (5) for determining a date of childbirth by analysing vascular activity of a pregnant person during pregnancy, the system comprising at least the following components: A wearable device (1) including a first sensor system (101) configured to be worn in contact with the skin of the pregnant person, wherein the wearable device (1) is further configured to detect vascular activity, such as heartbeats of the pregnant person, and to provide sensor signals indicative for the detected vascular activity; An analysing module (13, 30, 40) configured and arranged to process the sensor signals of the first sensor system (101), wherein the analysing module (13, 30, 40) is configured and arranged to determine from the sensor signals a date of childbirth. The invention further relates to a method for determining a date of childbirth by analysing vascular activity of a pregnant person.

The present invention relates to an electronic system and a method for determining the date of delivery for a pregnant person. In particular, the present invention relates to an electronic system and method for predicting the date of childbirth using a wearable device with a non-invasive sensor system.

The length of a pregnancy or gestational length is generally calculated as 40 weeks (or 280 days) after the first day of the last menstruation. However this date is at most an estimation. Only 4% of deliveries actually happen precisely on that date and only 70% of deliveries happen within 10 days of this calculated date [1]. The rational for this calculation is that ovulation is assumed to occur two weeks after the first day of the menstrual cycle (i.e. the day when the period begins) and the gestational length is calculated as 38 weeks from conception.

The length of a woman's cycle can, however, be variable so that the calculation method mentioned above is often incorrect. A more accurate method to calculate the delivery date would be to base it on ovulation. As conception has to occur within a time window of 24 hours after ovulation, the day of ovulation can be taken as an estimation of the conception date. One way of estimating the day of ovulation is by serial measurement of basal body temperature, as basal body temperature increases slightly after ovulation. Another way of identifying the day of ovulation is with hormone-based urine tests. Over-the-counter ovulation tests detect the release of a large amount of LH (luteinizing hormone) around 24 hours before ovulation. Monoclonal antibodies directed specifically against LH are used to selectively detect the elevated LH levels in urine and thereby determine the day of ovulation. Ovulation can also be detected with serial ultrasound around the time of expected ovulation. The examiner follows a maximal increase and subsequent decrease in the follicle size and then assumes that ovulation has occurred. However, the reduction in the follicle can often be misinterpreted for a number of reasons, for example the post ovulatory follicle can fill with fluid and thus be falsely interpreted as still being pre-ovulatory. Therefore, it is not surprising that the delivery date is often incorrect.

Even when accounting for the fact that ovulation does not always occur on day 14 of the menstrual cycle, a study by the US National Institute of Environmental Health Sciences found that the gestational length range was 37 days [2]. Any pre-term births were not taken into account for this calculation. Even though the sample size analysed was small, the results give a good indication that the calculated due date is far from being accurate.

There is an unmet need for a more accurate prediction of the date of delivery. A better knowledge of the date of delivery would make planning easier for women, her partner and family but also for her midwife, health care practitioners, obstetricians and the hospitals and thus has the potential to make birth safer.

For women and their families, a precise prediction is desirable also for organizational reasons, e.g. organizing well in advance their professional and private life. The better prediction of the “true” day of birth can also provide valuable information for planning the date for a caesarean section.

According to the British National Institute for Health and Care Excellence (NICE) Clinical Guidelines on Caesarean Sections, the risk of respiratory problems is increased in babies that are born by caesarean section prior to the onset of labour. As this risk significantly decreases after 39 weeks gestational age, the guidelines recommend that planned caesarean sections should not be carried out before the 39 gestational week (see chapter 1.4.1.1 of CG132, published November 2011, last updated August 2012, accessed at https://www.nice.org.uk/guidance/cg132 on Apr. 1, 2019)

Waiting until gestational week 39, however, bears the risk that the onset of labour occurs spontaneously before week 39, thereby resulting in an unplanned caesarean section [3]. Unplanned caesarean sections have a higher risk compared to planned caesarean sections. For example, the mother needs to reach the hospital, preferably with a neo natal facility, with reasonable amount of time prior to the procedure that all measures can be made to reduce neo natal complications. While in urban areas neo natal facilities typically are available, this is often not the case for women giving birth in rural areas. Therefore, it would be a great advantage for the woman and her health care practitioner to know about the risk of giving birth before week 39.

WO 2007110625 A2 describes a device and method for the prediction of onset of labour. The measurement and data analysis are based on electromyographic readings of the subject. The data analysing means comprises a means adapted to produce a result indicative an electromyographic parameter known to vary cyclically in the period preceding labour, a data register to store previous results, and a data comparator to compare a currently generated result with previous results and thereby to identify a peak in the said parameter.

WO 2012021944 A1 describes methods and kits for predicting the onset of labour by calculating the ratio of a least two hormone levels. The prediction method is purely based on hormonal levels determined in a fluid or tissue sample. Physiological parameters, such as heart rate e.g., are not used for calculating the onset of labour.

US 2017/0224268 A1 discloses a system for determining a labour state by analysing the uterine muscle contractions with a portable module comprising at least one physiological sensor, for determining a physiological signal indicative of muscle contractions. The system might comprise detecting the heart rate for determining a labour state in a woman. The system however is not capable to predict any date of childbirth prior to the detection of labour-dependent muscle contractions. However, once labour sets, the determination of a date of childbirth is almost trivial, as it is imminent.

JP 2008011916 A discloses an alarm system for veterinary use configured to issue an alarm when delivery of a farm animal is imminent based on a heart rate measurement of the farm animal. The system is configured to detect the decrease in heart rate approximately 10 minutes before delivery—that is during labour of the farm animal, but not for a longer time period. The problem of a convenient and precise prediction of the date of childbirth before the onset of labour however remains largely unsolved.

Therefore it is an object of the current invention to provide a system and a method for precise non-invasive determination of a date of childbirth before the onset of labour.

The problem is solved by an electronic system having the features of claim 1. Advantageous embodiments are disclosed in the dependent claims.

According to claim 1, the electronic system comprises at least the following components
a wearable device including a first sensor system configured to be worn in contact with the skin of the pregnant person, wherein the wearable device is further configured to detect vascular activity, such as heart beats, a heart rate or a blood flow of the pregnant person, and to provide sensor signals indicative for the detected vascular activity,
an analysing module configured and arranged to receive and to process the sensor signals of the first sensor system, wherein the analysing module is configured and arranged to determine from the sensor signals a date of childbirth.

The term “electronic system” particularly refers to a particularly computerized system comprising a plurality of components, wherein the components are not necessarily physically connected with each other.

The term “computerized” particularly refers to a system or a device comprising one or more processors operable or operating according to one or more programs.

The term “pregnant person” particularly refers to a pregnant woman.

The term “wearable device” particularly refers to a device that has a weight and dimension that allow a person to carry the device for essentially any time interval.

The wearable device is particularly a body-wearable device having adjustment means to fix the device to a body part of the person.

The wearable device is particularly a wrist-wearable device having adjustment means, such as an adjustable wrist band to fix the device to a wrist or a joint of the person.

The wearable device can be any item that has contact to the skin, such as but not limited to a watch-like device worn on the wrist, a bracelet, a cuff worn on the body, a ring or a device or clamp worn on the fingertip. A wearable device can also relate to components being integrated in a shirt or other garment.

The skin contact of the wearable device is particularly necessary for detecting the vascular activity.

The term “vascular activity” particularly refers to processes of the vascular system in response to or in connection with heart beating, such as pulse, blood flow, blood pressure etc.

The wearable device is particularly configured to detect heart beats, a heart rate, a blood flow and/or a pulse.

For this purpose the wearable device comprises the first sensor system.

The first sensor system comprises a sensor for detecting the vascular activity. The sensor system can also comprise a processing module in order to process the detected signals, such as to determine and output a sensor signal, comprising information on a feature of the vascular activity, such as a heart rate, heart beats or a (varying) blood flow rate.

According to an embodiment of the invention, the first sensor system is particularly configured to perform a photoplethysmography (PPG). Thus, the first sensor system is particularly an optical sensor system.

Alternatively or additionally, the first sensor system configured to detect an electro cardiogram signal (ECG). Either the first sensor system or the analysing module can be configured to process the sensor signals such as to calculate a heart rate or other parameters as mentioned previously.

For this purpose, it is understood by the person skilled in the art that the determination of the date of childbirth can not only be based on the heart rate extracted from a PPG signal but can also be based on other features extracted from the PPG signal directly, its first and/or second derivative. Such features are for example but not limited to: AC and DC components, rise time, amplitude, shape, pulse area, Peak-to-Peak Interval, rising edge of the pulse (anacrotic), the falling edge of the pulse (catacrotic), dicrotic notch, as well as features from the second derivative wave of the PPG signal called acceleration photoplethysmogram (APG), as for example described in [4].

Such PPG features are also comprised by the term “feature of the vascular activity” in the context of the current specification. The analysing module comprises at least one processor for processing the sensor signals and determining the date of childbirth.

According to another embodiment of the invention, the analysing module is comprised in the wearable device.

According to another embodiment of the invention, the analysing module is comprised in a computerized external device, such as a mobile phone or another mobile and portable device, such as a tablet, a laptop computer, and/or a server.

The analysing module can be distributed between several computerized devices, such that particularly predefined processing steps are performed on different computerized devices.

According to one embodiment of the invention, the analysing module comprises as computer program with computer program code that, when executed on the analysing module, causes the analysing module to determine, particularly calculate the date of childbirth.

According to another embodiment of the invention, the analysing module comprises electronic circuits that are hard-wired for determining the date of childbirth.

Particularly in contrast to other “smart devices” known in the art, the analysing module according to the invention is configured to determine the date of childbirth from the sensor signals.

According to another embodiment of the invention, the analysing module is arranged and configured to determine a length of pregnancy of the pregnant person by analysing the sensor signals of the first sensor system.

According to another embodiment of the invention, the analysing module is configured and arranged to determine from the sensor signals of the first sensor system a date of childbirth by determining and evaluating a feature of the vascular activity such as a heart rate from the sensor signals of the first sensor system.

It is noted that the term “heart rate” and “pulse” are used interchangeably in this context.

It surprisingly turns out that by analysing the heart rate, particularly a temporal course of heart rates of the pregnant person, particularly over a time period of several days to weeks it is possible to determine the date of childbirth with high accuracy.

The heart rate can be detected by the first sensor system as elaborated above, e.g. by means of PPG.

The temporal course of heart rate particularly the temporal course of a filtered set of heart rates (as elaborated the following embodiment and examples), is indicative of the date of childbirth. The determination of the date of childbirth for example can be facilitated by means of comparison to model data acquired from a statistical number of pregnant persons with known dates of childbirth. The comparison can for example be done by means of a trained classifier, as elaborated in the following embodiments.

According to another embodiment of the invention, the analysing module is configured and arranged to determine the date of childbirth from the sensor signals of the first sensor system by determining from the sensor signals, particularly from the detected heart rate, an onset of a period of a decreasing heart rate of the pregnant person, particularly wherein the period of a decreasing heart rate is a period of a decreasing heart rate in the third trimester of the pregnancy of the pregnant person.

The term “period of decreasing heart rate” particularly refers to an interval of particularly consecutive time points for which a heart rate is determined, wherein the determined heart rate is decreasing within said period. For this purpose, the heart rate can be monitored periodically, particularly at predefined time points. From the plurality of determined heart rates a filtered set of heart rates can be calculated to select a statistical subset of heart rates. Particularly for the determination of the onset of the period of a decreasing heart rate said filtered set of heart rates can be used.

In the context of the specification, the term “heart rate” particularly comprises such a filtered set of heart rates.

The filtered set of heart rates might comprise an average, a cumulant and/or a statistical set of heart rates, such as a specific percentile of the heart rate distribution, which is for example calculated for each day, and/or for each week.

The electronic system being configured to detect said onset of the period of a decreasing heart rate allows prediction of the date of childbirth with high accuracy, even if monitoring of the heart rate is started weeks after conception, as the decrease of interest happens in the third trimester of pregnancy.

The onset of the period of decreasing heart rates is particularly characterized in that in two, particularly three or more consecutive time points at which the heart rate or for which the filtered set of heart rates has been generated, the heart rate (or its filtered statistical value from the filtered set of heart rates) is decreasing.

Particularly, a decrease of heart rates of three to four beats per minute from a maximum heart rate, within a time span of 5 to 10 weeks can be indicative of the period of decreasing heart rates.

The identification of the onset of the period of a decreasing heart rate can for example be facilitated by means of a trained classifier, an artificial neural network or other methods known from machine learning.

Therefore, according to another embodiment of the invention, the onset of the period of decreasing heart rate is determined by a trained classifier particularly by means of an artificial neural network, particularly wherein the trained classifier is executed on the wearable device, particularly wherein the wearable device is configured to execute the trained classifier.

Alternatively to the onset of the period of decreasing heart rate, it is also possible to identify other specific features of the temporal course of the heart rate or the filtered set of heart rates during pregnancy, such as a local maximum of the heart rate preceding the onset of the period of decreasing heart rate. Particularly, said local maximum is not before the 25^{th} week of pregnancy, particularly wherein said local maximum is within the third trimester of pregnancy.

According to another embodiment of the invention, the analysing module is configured and arranged to identify said specific features of the temporal course of the heart rate,

According to another embodiment of the invention, the onset of the period of decreasing heart rate is determined by detecting decreasing heart rates determined for three or four consecutive days, particularly wherein said days are not before the 25t^{h} week of pregnancy, particularly wherein said days are within the third trimester of pregnancy.

According to another embodiment of the invention, the onset of the period of decreasing heart rate is after more than 25 weeks of pregnancy.

According to another embodiment of the invention, the onset of the period of decreasing heart rate is more than 10 days, particularly more than 20 days, more particularly more than 30 days before the date of child birth.

According to another embodiment of the invention, the date of child birth is predicted more than 10, 20 and/or 30 days before the estimated date of child birth.

According to another embodiment of the invention, the wearable device, particularly the analysing module is configured to predict the date of child birth more than 10, 20 and/or 30 days before the estimated date of child birth, particularly by determining the onset of the period of decreasing heart rates, more particularly by extrapolating form said onset of the period of decreasing heart rates to an estimated date of child birth.

Particularly, the system is only configured to determine the date of child birth with an accuracy of 12 hours or worse from the determined onset of the period of decreasing heart rate.

According to another embodiment of the invention the wearable device comprises a second sensor system configured to detect a second parameter other than the vascular activity of the pregnant person, and wherein the analysing module is configured to receive and process the sensor signals of the second sensor system, wherein the analysing module is further configured and arranged to determine the date of childbirth from the sensor signals of the first sensor system and the second sensor system.

The second sensor particularly detects another parameter of the pregnant person, such as body temperature, and/or bioimpedance.

The combination of a second parameter increases the robustness of the electronic system and allows for higher prediction accuracy with regard to the date of childbirth.

According to another embodiment of the invention, the second sensor system is configured and arranged to detect an acceleration of the wearable device, particularly wherein the second sensor system comprises or is an accelerometer or an inertial measurement unit (IMU).

This allows the electronic system to detect phases of rest of the pregnant person, such that sleep phases can be detected. During sleep phases the heart rate can be detected and shows less inter-day variability, as the person is at rest.

According to another embodiment of the invention, the electronic system is configured to detect sleep phases by evaluating acceleration data provided by the second sensor system.

According to another embodiment of the invention, the system is configured to only evaluate heart rates that are measured during sleep, particularly during selected sleep phases, more particularly during REM-phase or non-REM phases.

From the combination of detected heart rates and acceleration (and/or body temperature) the electronic system is able to also detect whether the person wears the wearable device, such that the sleep phases can be reliably detected.

The term “sleep phase” particularly refers to times at which the pregnant person is asleep.

According to another embodiment of the invention, the wearable device comprises a clock module, for determining a time of day.

According to another embodiment of the invention, the electronic system is configured to measure the heart rates at predefined times, e.g. at night or during a specific time at night.

According to another embodiment of the invention, the electronic system is configured to receive information on sleep cycle of the pregnant person, wherein the system is configured and arranged to measure the heart rates only at selected sleep cycle phases, e.g. during a REM-phase (rapid eye movement phase) or a non-REM phase. In order to detect and distinguish selected sleep cycle phases, the system is particularly configured and arranged to determine a heart rate variability.

The heart rate variability can for example be calculated by determining the so-called “Root Mean Square of Successive Differences” (RMSSD) from the recorded vascular activity data. However, there are a plurality of other parameters and features comprised by the vascular activity data that can be used to determine the heart rate variability.

The information about the sleep cycle can be provided by a smart device, such as a smart phone that is configured to detect the sleep cycle.

According to another embodiment of the invention, the analysing module is configured to detect sleep phases of the pregnant person by determining and evaluating a heart rate variability from the sensors signals of the first sensor system and/or by evaluating the acceleration determined by the second sensor system.

For this purpose the electronic system is configured to determine the heart rate several times or continuously during 24 h.

The heart rate variability estimates the time intervals between single consecutive heart beats and can be determined from the vascular activity data as described above.

In combination with the second sensor system, this embodiment allows for reliable sleep phase detection.

According to another embodiment of the invention, the electronic system comprises a data storage, wherein the electronic system is configured and arranged to store the sensor signals of the first and/or the second sensor system and/or the determined heart rate, a filtered set of heart rates and/or heart rate variability of the pregnant person in the data storage.

This allows the electronic system to evaluate the heart rates over many days and weeks.

The electronic system can be configured to store all parameters, information and/or signals that have been provided to or determined to the electronic system.

According to another embodiment of the invention, the analysing module is configured and arranged to determine the date of childbirth by evaluating the sensor signals of the first and/or the second sensor system and/or the determined heart rate, and particularly the heart rate variability stored in the data storage, particularly by determining the onset of the period of a decreasing heart rate from the stored data i.e. sensor signals, heart rate and heart rate variability.

Moreover, the analysing module can be configured to process information stored on the data storage, such as information relating to a sleep phase or a sleep cycle.

According to another embodiment of the invention, the analysing module is configured and arranged to compare a temporal course particularly extending over a plurality of days, particularly at the onset of the period of decreasing heart rate of the determined heart rate (or the filtered set of heart rates) of the pregnant person, to model data and to determine from said comparison a date of childbirth.

This embodiment and its advantages has been in large parts discussed in previous embodiments.

The electronic system is particularly configured and arranged to determine the date of childbirth based on heart rates or a set of filtered heart rates that have been acquired by the first sensors system at predefined time points during the day.

The term “time point” particularly refers to a time during day and/or to a waking state of the person.

According to another embodiment of the invention, the analysing module is configured and arranged to determine the date of childbirth, particularly the heart rate, the filtered set of heart rates, more particularly the onset of the period of a decreasing heart rate from sensor signals of the first and/or the second sensor system acquired during sleep phases, particularly during predefined sleep cycles of the pregnant person.

This embodiment allows for a robust and precise estimation of a heart rate with minimal influence of circumstantial parameters, such as the physical activity of the pregnant person.

According to another embodiment of the invention, the analysing module is configured and arranged to determine the date of childbirth from the sensor data of the first sensor comprising the lower 10th percentile of the heart rate acquired during sleep phases of the person.

The heart rate can be determined several times during a sleep phase or once every sleep phase.

The plurality of determined heart rates particularly acquired during the sleep phase(s) exhibit a heart rate distribution, e.g. a Gaussian distribution reflecting the frequency of a measured heart rate. Said distribution might be centred around a mean heart rate. From the heart rate distribution (which is not necessarily a Gaussian distribution) it is possible to identify the heart rates comprised by the lower 10^{th} percentile of heart rates.

The lower 10^{th} percentile on the one hand provides a stable and robust estimation of a resting pulse of the pregnant person and on the other hand excludes corrupt measurements that might have missed heart beats.

According to another embodiment of the invention, the analysing module is configured and arranged to determine the date of childbirth from the sensor data of the first sensor comprising the lower 20th percentile of the heart rate acquired during sleep phases of the person.

The problem according to the invention is furthermore solved by a method for determining a date of childbirth comprising the steps of
detecting a vascular activity, such as a heartbeat or a heart rate of a pregnant person,
providing sensor signals indicative for the detected vascular activity,
determining an onset of a period of decreasing heart rate, particularly in the third trimester of pregnancy from the sensor signals, and
predicting the date of childbirth from the determined onset of a period of a decreasing heart rate.

The terms and definitions provided for the embodiments related to the electronic sensor system apply also to the method according to the invention.

The method according to the invention allows for a non-invasive, reliable and robust determination of the date of childbirth.

According to an embodiment of the invention, the method comprises the steps of determining the date of childbirth from sensor signals acquired during sleep phases of the pregnant person.

This embodiment allows for a more consistent heart rate estimation devoid of any variations due to the person wake-activity, causing increased heart rates (e.g. physical exercise).

According to another embodiment of the invention, the sleep phases of the pregnant person are determined by evaluating a heart rate variability and/or sensor signals indicative of a resting state pregnant person, such as acceleration.

This embodiment allows distinguishing active phases and resting phases of the pregnant person such as to obtain more reliable measurements.

This embodiment allows for reliable detecting sleep phases of the pregnant person, either by monitoring the heart variability, e.g. the variance of the heart rate over a fixed time interval and/or by evaluating movements or motions of the person that are reduced during sleep phases, particularly by means of an accelerometer, a GPS.

Additionally or alternatively, the heart rates can be estimated at fixed times of the day, e.g. between 3 am and 4 am.

According to another embodiment of the invention, the method is executed on the electronic sensor system according to the invention, particularly wherein the pregnant person is wearing the wearable device.

According to another embodiment of the invention, the predicted date of childbirth or a symbol being indicative of the date of childbirth or its approach is displayed to the pregnant person, e.g. on a mobile device such as a mobile phone.

For this purpose the electronic system can comprise a mobile device, such as a mobile phone, a smart phone, a smart watch that is configured to communicate with the wearable device such as to exchange data.

The date of childbirth can be provided and/or displayed in form of a symbol indicative of a statistical confidence in the predicted date of childbirth, e.g. symbolizing a 70% probability for a specific date, and a 20% probability for the following day.

According to another embodiment of the invention, the heart rate is measured at least once every 24 h and wherein each week an average of 7 days from the heart rate measurements is calculated, particularly from the lowest 20^{th} or 10^{th} percentile of the heart rate distribution, wherein a temporal course from the said average heart rate is generated, wherein the onset of a period of decreasing heart rates is determined from said temporal course.

The problem is furthermore solved by a computer program comprising instructions to cause the electronic system to execute the steps of the method according to the invention.

According to another embodiment of the invention, the computer program is executed on the analysing module or a plurality of analysing modules being distributed on the wearable device, and at least one external computer.

Particularly, exemplary embodiments are described below in conjunction with the Figures. The Figures are appended to the claims and are accompanied by text explaining individual features of the shown embodiments and aspects of the present invention. Each individual feature shown in the Figures and/or mentioned in said text of the Figures may be incorporated (also in an isolated fashion) into a claim relating to the device according to the present invention.

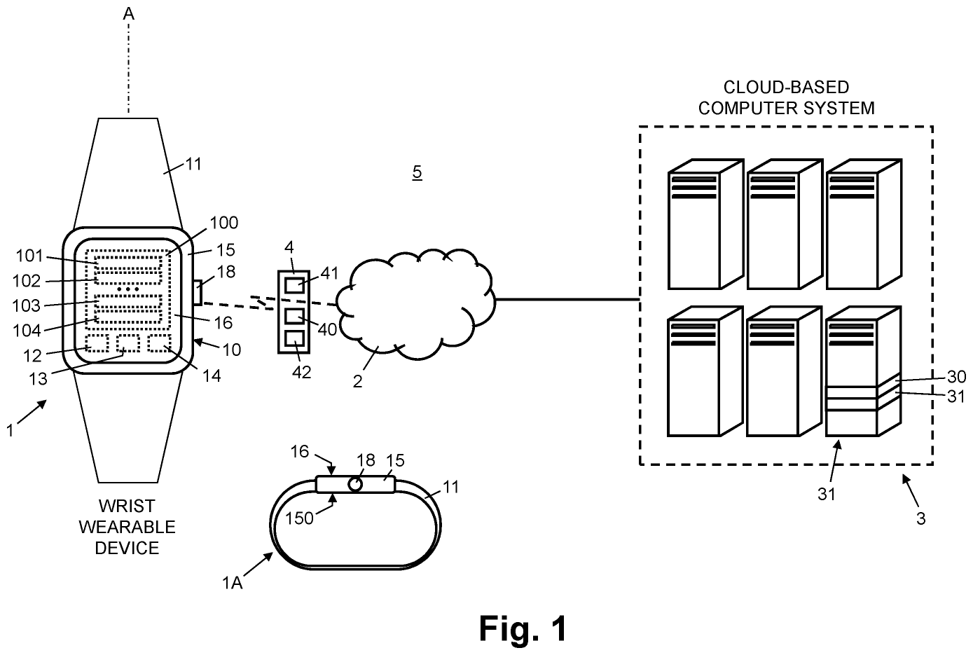

FIG. 1: shows a schematic illustration of an electronic system according to the invention for detecting the heart rate and further parameters related to the pregnancy of a person, the electronic system comprising a wearable device, in particular a wrist-worn bracelet, with an analysing module comprising a processor in the wearable device and/or in an external system.

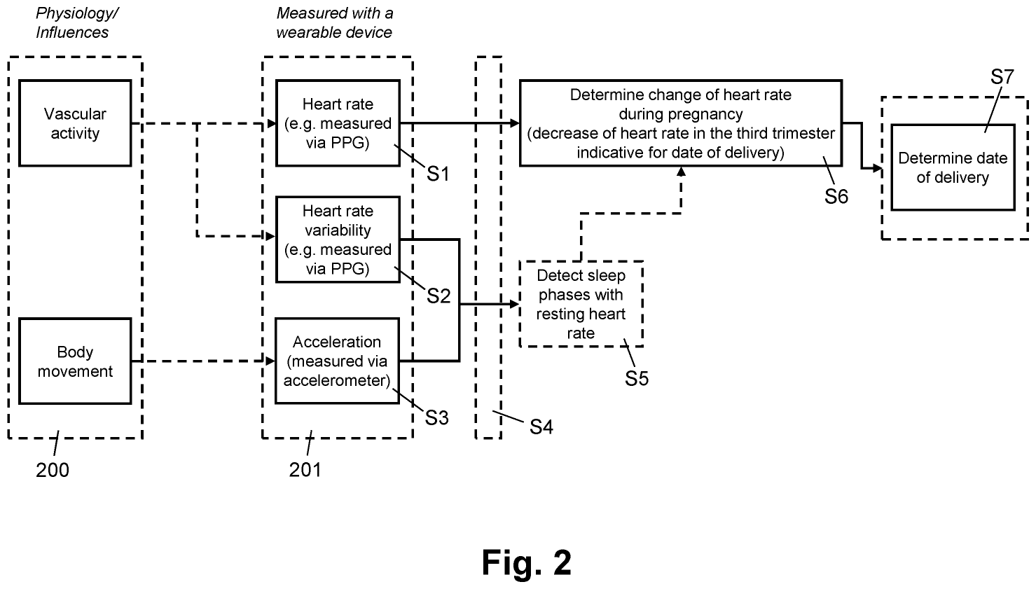

FIG. 2: shows a flow diagram schematically illustrating an exemplary sequence of steps for determining the date of childbirth by analysing the heart rate during pregnancy.

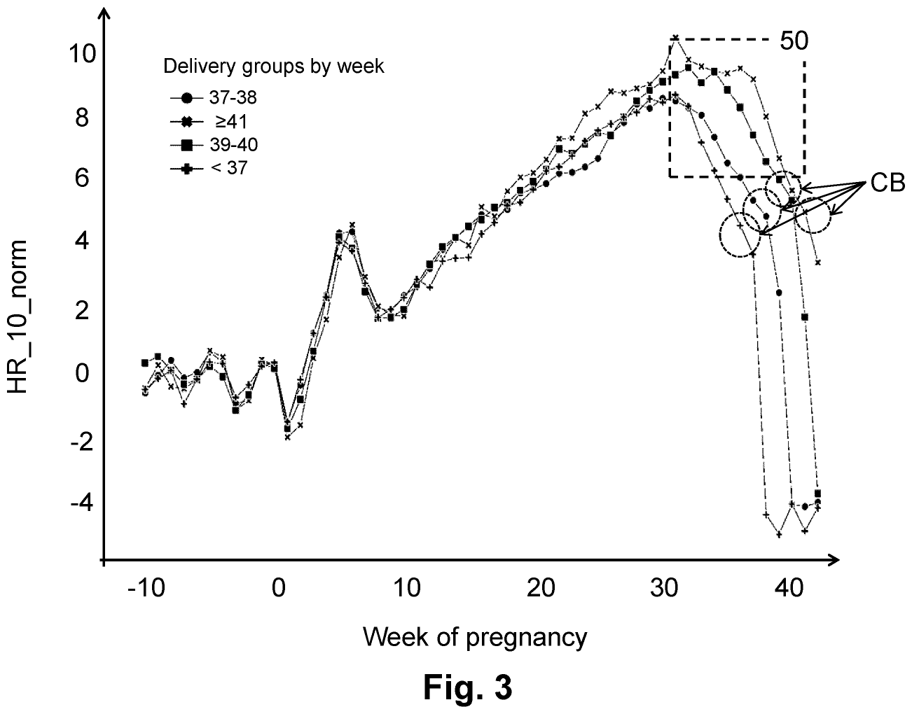

FIG. 3: shows a line graph illustrating the 10^{th} percentile of a heart rate distribution referenced to a heart rate prior conception (weeks −10 to 0) for of a group of pregnant women over the weeks of pregnancy. The data is grouped according to the delivery date in: deliveries before 37 weeks (indicated by a line with a plus sign “+”), deliveries between 37-38 weeks (indicated by a line with solid circles), deliveries between 39-40 weeks (indicated by a line with solid squares) and deliveries later than 41 weeks (indicated by a line with “x”) of pregnancy.

### DETAILED DESCRIPTION OF THE PREFERRED EMBODIMENTS

FIG. 1 shows an electronic system 5 for detecting a change in a vascular activity, such as the heart rate. The electronic system 5 comprises a wearable device 1 and an analysing module comprising one or more processors 13, 30, 40 in the wearable device 1 and/or in an external system. Reference number 3 refers to a computer system, e.g. a server, a cloud-based system, comprising one or more computers 31 with one or more processors 30 and one or more data storage systems 31. The computer system 3 or its processors 30, respectively, are connected to the data storage system 31 and configured to execute various functions, as will be explained in more detail. The data storage system 31 for example comprises a RAM, a flash memory, hard disks, data memory, and/or other data storages.

In FIG. 1, reference numeral 4 refers to a communication device, in particular a mobile communication device, e.g. a mobile phone, a cellular telephone, a tablet or laptop computer, comprising one or more processors 40, a data storage 41, and data entry elements 42. The processors 40 are connected to the data storage 41 and configured to execute various functions, as will be explained in more detail. The data storage 41 comprises a RAM, a flash memory, a data memory, and/or other data storage. The data entry elements 42 for example comprise one or more keys, a keyboard, and/or a touch sensitive screen enabling the user to enter data and/or event indications.

In FIG. 1, reference number 1 refers to a wearable device, e.g. a wrist wearable device, specifically a wrist wearable electronic device. Reference numeral 1A refers to a cross-sectional view of the wearable device 1 along central axis A. The wearable device 1 includes a fixation system for attaching the wearable device 1 on the body of a user, specifically, for attaching the wearable device 1 and particularly the first sensor system 101 in contact with the skin of the user, such that heart beats can be detected by the first sensor system 101; in the embodiment shown in FIG. 1, the wearable device 1 comprises a wrist band 11 and a device body 10 attached to or integrated in the wrist band 11. The wristband 11 is implemented as a watchstrap, a watchband, a bracelet, a cuff, or the like. The device body 10 comprises a housing 15 and an optional display 16 integrated in the housing 15.

As illustrated schematically in FIG. 1, the wearable device 1 comprises several sensor systems 100, including the first sensor system 101 with optical sensors configured to generate photoplethysmography (PPG) signals for measuring heart signals, heart rate, heart rate variability, perfusion, and breathing rate. For example, sensor system 101 comprises a PPG-based sensor system for measuring heart signals, heart rate and heart rate variability as described in Simon Arberet et al., “Photoplethysmography-Based Ambulatory Heartbeat Monitoring Embedded into a Dedicated Bracelet”, Computing in Cardiology 2013; 40:935-938, included herewith by reference in its entirety.

In an embodiment, the sensor systems 100 further include a second sensor system 102 with one or more accelerometers for measuring body movements (acceleration).

In an embodiment, for the purpose of sleep phase analysis the accelerometers are implemented in combination with the PPG-based sensor system, as described in Philippe Renevey et al., “Photoplethysmography-based bracelet for automatic sleep stages classification: Preliminary Results,”, IASTED 2014, Zurich, Switzerland, included herewith by reference in its entirety.

The sensor systems 100 further include a temperature sensor system 104 for measuring the user's temperature; specifically, the user's skin temperature; more specifically, the wrist's skin temperature. The temperature sensor system 104 comprises one or more sensors, including at least one temperature sensor and in an embodiment one or more additional sensor(s) for measuring further parameters like a perfusion, a bio-impedance and/or a heat loss for determining the user's temperature.

Depending on the embodiment, the sensor systems 100 further can include a bio-impedance sensor system 103 with an electric impedance or conductance measuring system. The optical sensors 101, the bio-impedance sensor system 103, and the temperature sensor system 104 are integrated in a housing 15 of the wearable device 1 and are arranged on a rear side 150 of the wearable device 1, e.g. opposite of an optional display 16 of the wearable device 1, facing the user's skin in a mounted state of the wearable device 1. In the mounted state, when the device 1 is actually attached and worn, e.g. on the wrist, just as one would wear a watch, the rear side 150 of the wearable device 1 or the rear side 150 of its housing 15, respectively, is in contact with the skin, e.g. the skin of the wrist, i.e. the optical sensors 101, the bio-impedance system 103, and the temperature sensor system 104 touch the skin or at least face the skin, e.g. the skin of the wrist.

The wearable device 1 further comprises a data storage 12, e.g. a data memory such as RAM or flush memory, and an operational processor 13 connected to the data storage 12 and the sensor systems 100. The processor 13 comprises an electronic circuit configured to perform various functions.

As illustrated in FIG. 1, in an embodiment, the wearable device 1 further comprises a communication module 14 connected to the processor 13. The communication module 14 is configured for data communication with an external system 3, 4, that is separated from the wearable device 1, i.e. a computerized system that is arranged in a different housing than the wearable device 1. Depending on the embodiment and/or configuration, the external system is a remote computer system 3 or a mobile communication device 4. Accordingly, the communication module 14 is configured for data communication with the remote computer system 3 via a network 2 and/or with the mobile communication device 4 e.g. via a close range communication interface. The network 2 comprises a mobile radio network such as a GSM-network (Global System for Mobile communication), a UMTS-network (Universal Mobile Telephone System), or another mobile radio telephone system, a wireless local area network (WLAN), and/or the Internet. For example, for close range communication, the communication module 14 comprises a Bluetooth communication module, e.g. a Low Energy Bluetooth module, or another close range communication module configured for direct data communication with the external mobile communication device 4. In an alternative embodiment, the mobile communication device 4 is configured to facilitate the data communication between the wearable device 1 and the remote computer system 3, e.g. by relaying the measurement data from the wearable device 1 via the network 2 to the remote computer system 3, for processing. Although not illustrated, the wearable device 1 further comprises a timer module configured to generate current time and date information, e.g. a clock circuit or a programmed timer module. The timer module is further configured to generate time stamps including the current time and date. As further illustrated in FIG. 1, the wearable device 1 further comprises one or more data entry elements 18 enabling the user to enter data and/or event indications. Depending on the embodiments, data entry elements 1B comprise data entry buttons, keys and/or rotary selection switches.

In FIG. 2 box 200 relates to physiological parameters and other factors, including vascular activity and body movement of the pregnant person, which are used for determining and predicting the date of giving birth with the electronic system according to the invention, particularly by using the first and the second sensor system 101, 102.

In FIG. 2 box 201 includes the detected parameters, i.e. the heart rate and a heart rate variability determined from the signals of the first sensor system 101 and the acceleration of the wearable device 1 as determined from the second sensor system 102.

As illustrated in FIG. 2, in step S1, the heart rate of the pregnant person wearing the wearable device 1 is measured using the wearable device 1. Specifically, in the state of the device 1 being worn, e.g. on the wrist, the processor 13 of the wearable device 1 reads or receives from the first sensor system 101 the current heart rate of the pregnant person. The processor 13 stores the heart rate (value) in the data storage 12 together with a time stamp, including the current time and date.

In (an optional) step S2, the heart rate variability of the pregnant person is measured using the wearable device 1. Specifically, in the state of the device 1 being worn, e.g. on the wrist, the processor 13 of the wearable device 1 reads or receives from the first sensor system 101 the current heart rate variability of the pregnant person. The processor 13 stores the heart rate variability (value) in the data storage 12 together with a time stamp, including the current time and date.

In step S3, the movement or acceleration, respectively, of the pregnant person is measured using the wearable device 1. Specifically, in the state of the device 1 being worn, e.g. on the wrist, the processor 13 of the wearable device 1 reads or receives from the second sensor system 102 the acceleration of the wearable device and thus the acceleration of the wrist of the pregnant person. The processor 13 stores the acceleration (value) in the data storage 12 together with a time stamp, including the current time and date. In some simplified embodiments, step S3 is omitted.

Preferably, the measurements of the heart rate, the heart rate variability, and the acceleration of the pregnant person are performed concurrently. The measurements of the first and the second sensor system 101, 102 are performed periodically, for example the first sensor system 101 uses the optical sensors to measure the heart rate and heart rate variability every couple of milliseconds. In an embodiment, the periodic measurements are limited to specific time intervals, e.g. during night time, when the pregnant person sleeps, such that the heart rate is measured during sleep phases only.

Depending on the embodiment and/or configuration, further processing of the detected heart rate, heart rate variability, and acceleration of the pregnant person is performed by the processor 13 of the wearable device 1 and/or by the processor(s) 30, 40 of the computer system 3 and/or the mobile communication device 4. In a case that involves processing by the processor(s) 30 of the computer system 3, the measured and time stamped values of the heart rate, heart rate variability, and acceleration are transmitted by the communication module 14 from the wearable device 1 via the network 2 to the computer system 3, as indicated by step S4 in FIG. 2, e.g. directly or via the mobile communication device 4 as a relay device. In a case that involves processing by the processor 40 of the mobile communication device 4, the measured and time stamped values of the heart rate, heart rate variability, and acceleration are transmitted by the communication module 14 from the wearable device 1 via the close range communication interface to the mobile communication device 4 where they are stored in the data storage system 41. In the computer system 3 and/or the mobile communication device 4, respectively, the received measurement values are stored securely assigned to the pregnant person, defined, for example, by a user identifier and/or a device identifier (for increased anonymity/privacy). Transmission of the time stamped measurements is performed periodically, for example; typically, the measurement data is transmitted less frequently than the measurements are taken, e.g. various time stamped measurement samples, taken at different times, are grouped and transmitted together by the wearable device 1 in a combined data transmission.

In step S5, the heart rate variability and the acceleration are used (by the processor 13 of the wearable device and/or by the processor(s) 30, 40 of the computer system 3 and/or the mobile communication device 4) to detect sleep phases with a resting pulse. Detecting sleep phases with resting pulse allows detecting the heart rate each night in the same state of activity of the pregnant person. The sleep phases are detected, for example, by combining the measurements of the heart rate variability and acceleration as described by Renevey et al. cited above. In a simplified embodiment, the sleep phase is determined without using the measured acceleration and the second sensor system at all, for example based on a user-defined sleep interval, e.g. between 3:00 am and 4:00 am.

In step S6, the processor 13 of the wearable device 1 and/or the processor(s) 30, 40 of the computer system 3 and/or the mobile communication device 4 detect changes of the heart rate, e.g. the resting pulse. In other words, the processor(s) 13, 30, 40 determine changes of the heart rate, i.e. changes in the duration of the interval between individual heart beats, respectively, that occur during the detected sleep phases with resting pulse. Specifically, the processor(s) 13, 30, 40 determine the points in time when the resting pulse rate in the third trimester of the pregnancy starts decreasing.

In a simplified embodiment, the wearable device 1 and/or the processor(s) 30, 40 of the computer system 3 and/or the mobile communication device 4 detect changes of the pulse during pregnancy, without a limitation to a detected sleep phase, but at a specific point in time, e.g. during the night, for example based on a user-defined sleep interval, e.g. between 3:00 am and 4:00 am.

As seen in FIG. 3, determination of the date of delivery can be calculated as the date of onset of a decrease in heart rate in the third trimester plus 5.5 weeks+/−0.5 weeks.

The date of childbirth CB is encircled for the different groups.

The heart rate parameter used in the analysis is the 10^{th} percentile of the heart rate measurements of the full night per subject per night. Weekly averages are then calculated per subject and these are further normalized by subtracting the average of this parameter prior to conception, to account for differing base lines. Subjects have been sorted into delivery week groups and the average for each group and week is displayed. A total of 644 pregnancies were used for this analysis. 66 are in delivery week group “<37”, 193 in “37-38”, 339 in “39-40” and 46 in “>=41” as explained above.

As can been seen in all groups after conception the heart rate increases to a local maximum around week 5-6 followed by a short period of a decrease. Starting from week 10, the heart rates in all groups increase approximately until week 30 after conception. Depending on the date of childbirth a period 50 of decreasing heart rates indicated by the boxed region follows to the maximum heart rate around week 30.

Obviously it is possible to differentiate between the decrease around week 10 and 30, simply by providing an approximate date of conception.

The electronic system and method is configured such that the onset of said period is detected and a prediction of the date of childbirth becomes possible.

From FIG. 3 it can be seen that for example in the group of pregnant persons that gave birth before week 37 the onset of the period 50 of decreasing heart rate shows earlier and remains steep, as compared to for example the group of pregnant women that gave birth in week 39 to 40.

The onset of the period 50 can be determined detecting three to four heart rates (or filtered sets of heart rates) estimated over a week, that are consecutively decreasing. Such a pattern gives a clear indication of an imminent childbirth.

Alternatively, the detection of said onset can be for example also facilitated by means of a trained classifier, such as an artificial neural network that is provided with the weekly heart rates.

The method and the electronic system allow for providing the pregnant person with a date of childbirth. In one embodiment the predicted date of childbirth is combined with information about a confidence value that indicates an estimated probability associated with the predicted date of childbirth.

For example when the heart rate decreases two weeks in a row around week 30, a date of child birth can be determined. However, as can be seen for example for the groups giving birth before week 37 and between week 37 and 38 (and even for deliver after week 41), the onset of the period 50 of decreasing heart rates is very similar such that an accurate prediction requires more measurement points. Nonetheless, a probability (confidence information) for each possible week of delivery can be given such that the pregnant person is able to put the determined date of childbirth in perspective.

The electronic system and the method according to the invention allows for a novel, reliable and non-invasive way of predicting the date of childbirth.

### REFERENCES

[1] Mongelli M., Wilcox M., Gardso J.; “Estimating the date of confinement: Ultrasonographic biometry versus certain menstrual dates”; American Journal of Obstetrics and Gynecology, 1996, Vol 174, 174:278-81.
[2] Jukic A. M., Baird D. D., Weinberg C. R., McConnaughey D. R., Wilcox A. J., “Length of human pregnancy and contributors to its natural variation”; Human Reproduction, 2013, Vol. 28, 2848-2855; doi:10.1093/humrep/det297
[3] Wilmink F. A., Pham C. T., Edge N., Hukkelhoven C. W. P. M., Steegers E. A. P., Mol B. W., “Timing of elective pre-labour caesarean section: A decision analysis”, Australian and New Zealand Journal of Obstetrics and Gynaecology, 2018, 1-7; DOI: 10.1111/ajo.12821
[4] Castaneda D., Esparza A., Ghamari M., Soltanpur C., Nazeran H., “A review on wearable photoplethysmography sensors and their potential future applications in health care”, International Journal of Biosensors & Bioelectronics, 2018, 4(4):195-202

1. An electronic system for determining a date of childbirth by analysing vascular activity of a pregnant person during pregnancy, the system comprising:
a wearable device including a first sensor system configured to be worn in contact with the skin of the pregnant person, wherein the wearable device is further configured to detect vascular activity, including a heart rate of the pregnant person, and to provide sensor signals indicative of the detected vascular activity; and
an analysing module configured and arranged to process the sensor signals of the first sensor system, wherein the analysing module is configured and arranged to determine from the sensor signals of the first sensor system a date of childbirth by determining from the sensor signals, the heart rate, and an onset of a period of a decreasing heart rate of the pregnant person.

2. The electronic system according to claim 1, wherein the analysing module is configured and arranged to determine from the sensor signals of the first sensor system a date of childbirth by determining and evaluating a feature of the vascular activity from the sensor signals of the first sensor system.

3. The electronic system according to claim 1, wherein the period of a decreasing heart rate is a period of a decreasing heart rate in the third trimester of the pregnancy of the pregnant person.

4. The electronic system according to claim 1, wherein the wearable device comprises a second sensor system configured to detect a second parameter other than the vascular activity of the pregnant person, and wherein the analysing module is configured to process sensor signals of the second sensor system, wherein the analysing module is further configured and arranged to determine the date of childbirth from the sensor signals of the first sensor system and the second sensor system.

5. The electronic system according to claim 4, wherein the second sensor system is configured and arranged to detect an acceleration of the wearable device, wherein the second sensor system comprises an accelerometer.

6. The electronic system according to claim 5, wherein the analysing module is configured to detect sleep phases of the pregnant person by determining and evaluating a heart rate variability from the sensor signals of the first sensor system and/or by evaluating the acceleration determined by the second sensor system.

7. The electronic system according to claim 6, wherein the electronic system comprises a data storage system, wherein the electronic system is configured and arranged to store the sensor signals and/or the determined heart rate and or the heart rate variability of the pregnant person in the data storage system.

8. The electronic system according to claim 7, wherein the analysing module is configured and arranged to determine the date of childbirth by evaluating the sensor signals, the determined heart rate, and/or the heart rate variability stored in the data storage system, by determining the onset of the period of a decreasing heart rate from the stored data.

9. The electronic system according to claim 2, wherein the analysing module is configured and arranged to compare a temporal course of the determined feature of the vascular activity to model data and to determine from said comparison a date of childbirth.

10. The electronic system according to claim 1, wherein the analysing module is configured and arranged to determine the date of childbirth based at least in part on the onset of the period of a decreasing heart rate from sensor signals acquired during sleep phases of the pregnant person.

11. The electronic system according to claim 1, wherein the analysing module is configured and arranged to determine the date of childbirth from the sensor data comprising the lower 10^{th} percentile of the heart rates acquired during sleep phases of the person.

12. A method for determining a date of childbirth comprising the steps of:
detecting a vascular activity of a pregnant person;
providing sensor signals indicative for the detected vascular activity;
determining an onset of a period of decreasing heart rate, particularly in the third trimester of pregnancy from the sensor signals; and
predicting the date of childbirth from the determined onset of the period decreasing heart rate.

13. The method according to claim 12, wherein the method further comprises the steps of determining the date of childbirth from sensor signals acquired during sleep phases of the pregnant person.

14. The method according to claim 13, wherein the sleep phases of the pregnant person are determined by evaluating a heart rate variability and/or sensor signals indicative of a resting state pregnant person, wherein the sensor signals include acceleration.

15. The method according to claim 12, wherein the method is executed on the electronic system comprising a wearable device including a first sensor system configured to be worn in contact with the skin of the pregnant person, and an analysing module configured and arranged to process the sensor signals of the first sensor system.
